(19) United States
(12) Patent Application Publication
(10) Pub. No.: US 2015/0152379 A1
(43) Pub. Date: Jun. 4, 2015

(54) TISSUE CULTURE METHOD FOR PRODUCING AGARWOOD

(71) Applicant: MINGDAO UNIVERSITY, Changhua County (TW)

(72) Inventor: Jen-Ping CHUNG, Changhua County (TW)

(73) Assignee: MINGDAO UNIVERSITY, Changhua County (TW)

(21) Appl. No.: 14/093,946

(22) Filed: Dec. 2, 2013

(51) Int. Cl. C12N 5/04

(52) U.S. Cl. CPC C12N 5/04

(57) ABSTRACT

A tissue culture method for producing agarwood comprises a tissue culture/regeneration step: providing an agarwood explant, implanting the agarwood explant in a container under an antiseptic state, adding culture media into the container, and cultivating the agarwood explant into an agarwood plantlet; a microbe inoculation step: placing a resin-inducing agent into the container, and letting the resin-inducing agent invade the agarwood plantlet; and a resin formation step: letting the agarwood plantlet secrete a secondary metabolite to counteract the resin-inducing agent, and letting the secondary metabolite deposit to form agarwood. The present invention can mass-produce agarwood in an indoor environment without being limited by weather or soil quality and affected by variables (such as insect pests) existing in the natural environment, The present invention can further shorten the production period and accelerate cost recovery. Therefore, the present invention can satisfy requirements of manufacturers.

### FIELD OF THE INVENTION

The present invention relates to agarwood, particularly to a method for artificially producing agarwood that is the most highly valuable resinous wood used as incenses, perfumes, traditional medicines and other products in the world market.

### BACKGROUND OF THE INVENTION

Agarwood trees (or agarwood in brief) have also been reported from species in several other genera within the same family. But only A. malaccensis (syn.A. agallocha) and A. crassna are more frequently associated with agarwood trees that produce resin to have a smell of agarwood. The so-called agarwood is the metabolite of the juice of these trees. The xyloid portion of the metabolite is called the agarwood. Agarwood has a pretty high density, totally or partly sinking into water.

Agarwood is a natural product. The formation of agarwood is while the core of an agarwood tree is wounded or infected by microbes (e.g. fungi or bacteria) the tree secretes intensively aromatic resin, and the resin merges with the xylem to form the so-called agarwood.

Normally, agarwood is burned to give scent, extracted to obtain spice/essence, added into alcohol, or carved into decorative works. The shade-dried agarwood can be used as Chinese herb medicine. Used medically, agarwood testes bitter and performs temperately, and thus can treat bellyache, stomachache, vomit and asthma.

Natural agarwood is rare and unlikely to satisfy demand. Therefore, there have been artificial-cultivation technologies to mass-produce agarwood. For example, a U.S. Pat. No. 6,848,211 disclosed a technology of “Cultivated Agarwood”, which stimulates the natural development environment of agarwood, wherein the agarwood tree is artificially wounded, and gas is filled into the wound to inhibit curing of the wound, whereby the agarwood tree can secrete resin persistently to form agarwood.

The abovementioned prior art needs 4-5 year old agarwood trees and has to fill gas into the wounds persistently during the production process. Therefore, the prior art needs to afforest a. large area of land and may suffer from damages caused by forces majeure, such as typhoons or weather variation. In other words, the prior art needs high initial investment, has a long investment recovery period, and takes a high risk. Hence, the prior art is hard to meet the requirements of manufacturers.

### SUMMARY OF THE INVENTION

The primary objective of the present invention is to provide a method for fast producing agarwood, accelerating cost recovery in agarwood production and lowering the risk of agarwood production, which is applicable to the plant factory in quality agriculture and satisfies requirements of manufacturers

To achieve the abovementioned objective, the present invention proposes a tissue culture method for producing agarwood, which comprises a tissue culture/regeneration step, a microbe inoculation step, and a resin formation step. In the tissue culture/regeneration step, provide an agarwood explant; next, implant the agarwood explant in a container; next, add culture media into the container; and then cultivate the agarwood explant into an agarwood plantlet. In the microbe inoculation step, place a resin-inducing agent into the container, and let the resin-inducing agent invade the agarwood plantlet. In the resin formation step, let the agarwood plantlet secrete a secondary metabolite to counteract the resin-inducing argent, and let the secondary metabolite deposit to form agarwood.

The present invention has the following advantages:

1. The present invention can mass-produce agarwood in an indoor environment without being limited by weather or soil quality and affected by variables (such as insect pests) existing in the natural environment.

2. The present invention can shorten the production period, reduce the initial investment, accelerate investment recovery and satisfy requirements of manufacturers.

### BRIEF DESCRIPTION OF THE DRAWINGS

FIG. 1 is a flowchart of a tissue culture method for producing agarwood according to one embodiment of the present invention;

FIG. 2 is a photograph of an agarwood explant according to one embodiment of the present invention;

FIG. 3 is a photograph of an agarwood plantlet according to one embodiment of the present invention; and

FIG. 4 is a photograph of an agarwood plantlet having been produced resin according to one embodiment of the present invention.

### DETAILED DESCRIPTION OF THE PREFERRED EMBODIMENTS

Below, the technical contents are described in detail with embodiments. However, it should be noted: these embodiments are only to exemplify the present invention but not to limit the scope of the present invention.

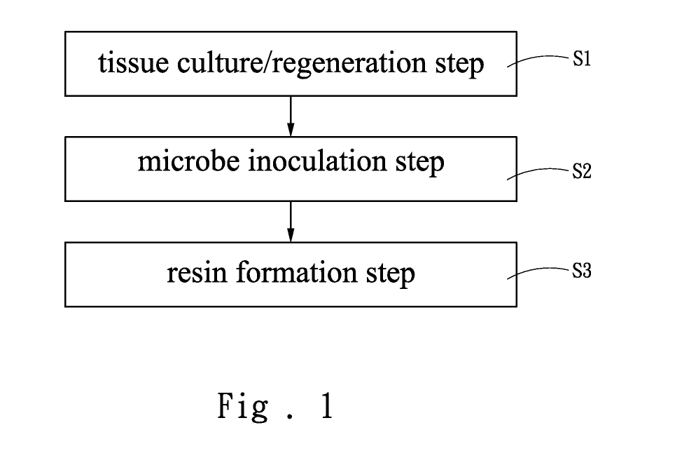

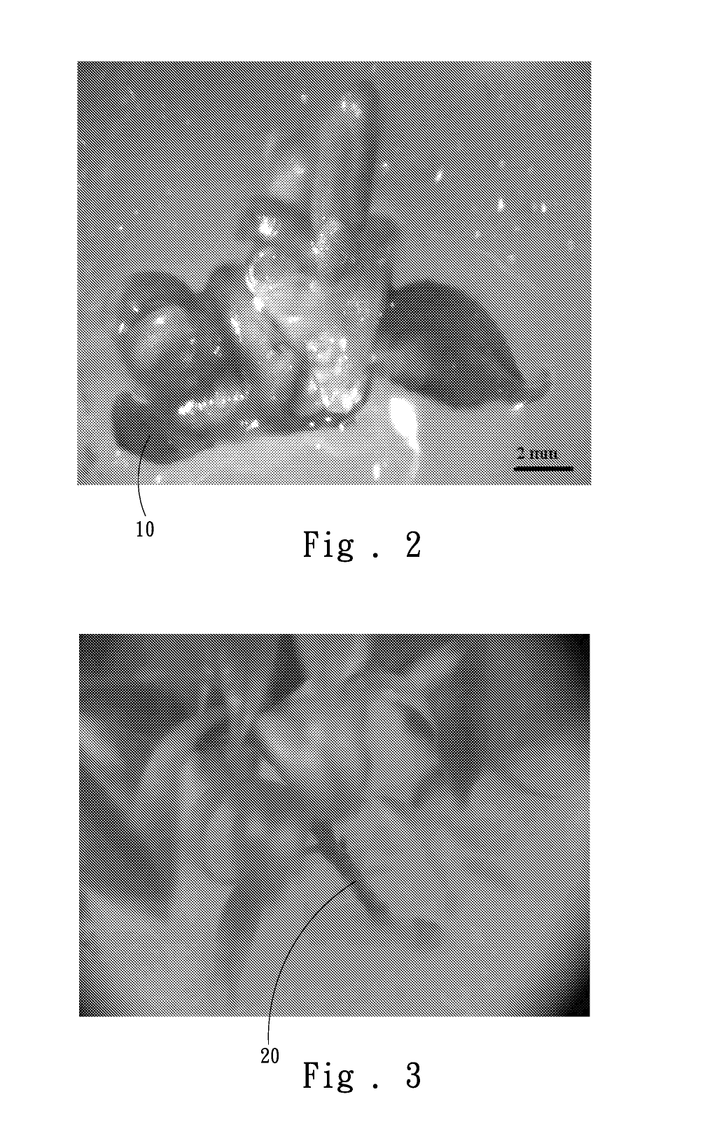

Refer to FIGS. 1-4. The present invention proposes a tissue culture method for producing agarwood, which comprises a tissue culture/regeneration step S1, a microbe inoculation step S2, and a resin formation step S3. In the tissue culture/regeneration step S1, provide an agarwood explant 10 (as shown in FIG. 2), wherein the agarwood explant 10 can be mass-produced in an asexual reproduction method; next, implant the agarwood explant 10 in a container (not shown in the drawings); next, add culture media into the container, wherein the container is airtightly sealed to insulate the interferences of the external environment, and wherein the culture media contain organic and/or inorganic compounds favoring the growth or regeneration of plants, such as culture plates for reproducing agarwood microbodies and proliferating twigs; and then, cultivate the cells and tissues of the agarwood explant 10 to grow into an agarwood plantlet 20 (as shown in FIG. 3). Normally, the agarwood plantlet 20 grows to a height of about 5 cm.

In the microbe inoculation step S2, place a resin-inducing agent into the container, and let the resin-inducing agent invade the agarwood plantlet 20. It is preferred that the resin-inducing agent is mixed with the culture media first, and then the mixture is placed into the container. The resin-inducing agent may be artificially cultivated. However, the resin-inducing agent is preferably extracted from the agarwood which has been produced resin.

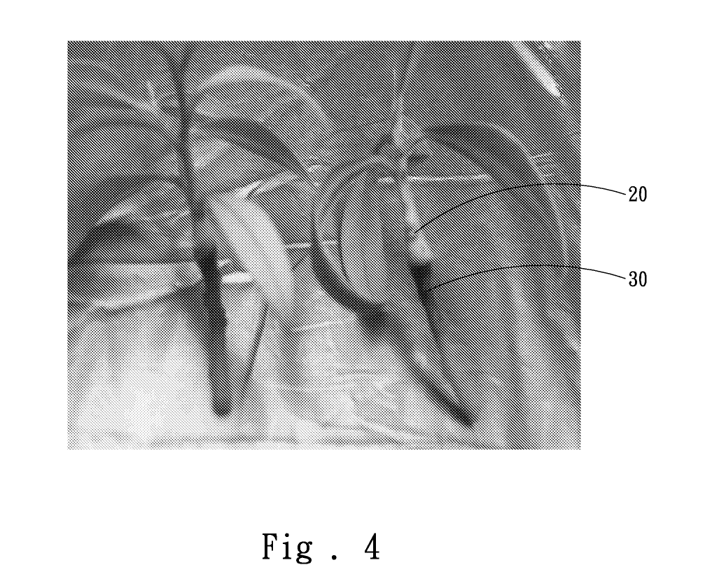

In the resin formation step S3, let the agarwood plantlet 20 secrete a secondary metabolite to counteract the resin-inducing agent, and let the secondary metabolite deposit to form agarwood 30, as shown in FIG. 4. During the resin formation step S3, the twigs of the agarwood plantlet are gradually blackened from bottom to top, and the blackened top of the twigs will continue growing. The resin formation process is regarded as completed while 70% of the agarwood plantlet 20 has blackened. In some embodiments, the agarwood plantlet 20 is allowed to grow for only 3-6 months so as to shorten the production period and achieve higher production efficiency.

In conclusion, the present invention can mass-produce agarwood explants in an asexual reproduction way and use culture Media to cultivate the agarwood explants into agarwood plantlets, and then place a resin-inducing agent to make the agarwood plantlets secrete the metabolite and deposit the metabolite to form agarwood. The present invention can mass-produce agarwood in an indoor environment without being limited by weather and soil and affected by variables existing in the natural environment, such as insect pest. The present invention can shorten the production period, reduce the initial investment, accelerate investment, recovery and satisfy requirements of manufacturers.

What is claimed is:

1. A tissue culture method for producing agarwood, comprising the steps of:
a tissue culture/regeneration step: providing an agarwood explant, implanting the agarwood explant in a container, adding culture media into the container, and cultivating the agarwood explant into an agarwood plantlet;
a microbe inoculation step: placing a resin-inducing agent into the container, and letting the resin-inducing agent invade the agarwood plantlet; and
a resin formation step: letting the agarwood plantlet secrete a secondary metabolite to counteract the resin-inducing agent, and letting the secondary metabolite deposit to form agarwood.

2. The tissue culture method for producing the agarwood according to claim 1, wherein in the tissue culture/regeneration step, the agarwood plantlet grows to a height of 5 cm.

3. The tissue culture method for producing the agarwood according to claim 1, wherein the container is airtightly sealed.

4. The tissue culture method for producing the agarwood according to claim 1, wherein the resin-inducing agent is extracted from the agarwood which has been produced resin,

5. The tissue culture method for producing the agarwood according to claim 1, wherein in the microbe inoculation step, the resin-inducing agent is mixed with the culture media to form a mixture, and the mixture is placed into the container.

6. The tissue culture method for producing the agarwood according to claim 1, wherein in the resin formation step, twigs of the agarwood plantlet are blackened to complete the resin formation step.

7. The tissue culture method for producing the agarwood according to claim 1, wherein in the resin formation step, the agarwood plantlet grows for 3-6 months.

8. The tissue culture method for producing the agarwood according to claim 1, Wherein the culture media are culture plates for reproducing agarwood microbodies and proliferating twigs.
